(19) United States
(12) Patent Application Publication
(10) Pub. No.: US 2007/0178507 A1
(43) Pub. Date: Aug. 2, 2007

(54) Method and apparatus for detection of molecules using nanopores

(76) Inventors: Wei Wu, Mountain View, CA (US); Zhiyong Li, Palo Alto, CA (US); Shih-Yuan Wong, Palo Alto, CA (US); Duncan Stewart, Menlo Park, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/655,388

(22) Filed: Jan. 19, 2007

Related U.S. Application Data
(60) Provisional application No. 60/763,634, filed on Jan. 31, 2006.

(51) Int. Cl. C12Q 1/68; G01N 33/53; C12M 3/00

(52) U.S. Cl. 435/6; 435/7.1; 435/287.2; 977/902; 977/924

(57) ABSTRACT

A molecular analysis device comprises a molecule sensor and a nanopore that passes through, partially through, or substantially near the molecule sensor. The molecule sensor may comprise a single electron transistor including a first terminal, a second terminal, and a nanogap or at least one quantum dot positioned between the first terminal and the second terminal. The molecular sensor may also comprise a nanowire that operably couples a first and a second terminal. A nitrogenous material that may be disposed on at least part of the molecule sensor is configured for a chemical interaction with an identifiable configuration of a molecule. The molecule sensor develops an electronic effect responsive to a molecule or responsive to a chemical interaction.

### CROSS-REFERENCE TO RELATED APPLICATION

This application claims the benefit of U.S. Provisional Patent Application Ser. No. 60/763,634, filed Jan. 31, 2006, for METHOD AND APPARATUS FOR DETECTION OF MOLECULES USING NANOPORES, the disclosure of which is incorporated by reference in its entirety.

### FIELD OF THE INVENTION

The present invention relates to analysis using nanoelectronic circuits. More particularly, the present invention relates to systems and methods for determining the chemical sequences of molecules using nanoscale transport systems, nanoscale sensors, and nanopores.

### BACKGROUND OF THE INVENTION

Determining the sequence of biological polymers, such as deoxyribonucleic acid (DNA) is, conventionally, a difficult and expensive process. However, with the rapid growth in nanotechnology, new methods may be devised to increase accuracy and speed while decreasing the cost of determining the constituent parts of biological polymers, such as protein, DNA, and ribonucleic acid (RNA).

Various methods have been developed for determining the chemical composition of portions of a DNA strand or the chemical composition of an entire DNA strand. One such method involves creating a micro-array with hundreds or thousands of patches of single stranded DNA (often referred to as probes) attached to various locations on a substrate, such as glass or silicon.

When using this detection method, the DNA to be examined is first transcribed into RNA. RNA is a chemical very similar to DNA that can encode the same information as DNA. The RNA can then be used to create single stranded DNA (ssDNA) copies of the RNA. Fluorescent molecules, also referred to as tags, are then bonded onto the new single stranded DNA molecules.

When the tagged ssDNA molecules are washed over the micro-array, they bond and stick to any of the ssDNA probes having a complementary gene sequence. Then, a light source exposing the micro-array causes the tagged DNA molecules stuck to the micro-array to fluoresce. The fluorescent glow can be detected and, based on where the various DNA tags were placed and their corresponding sequence, the sequence of the portion of the DNA stuck to that site can be determined.

Unfortunately, this process requires a significant number of chemical and optical steps to determine various portions of a DNA sequence. In addition, the detection is limited to the variety of DNA probes on the micro-array. Long probes with a large number of sequences can detect a significant match, but it becomes difficult to place every possible variation of long probes on a single micro-array. On the other hand, short probes may be incapable of detecting a desired long sequence.

Another detection method involves examining a polymerase chain reaction replication process. An RNA polymerase may attach to a DNA molecule and begin separating the DNA strand. The RNA polymerase then traverses along the DNA strand opening newer regions of the DNA strand and synthesizing an RNA strand matching the opened portions of the DNA. As the RNA polymerase traverses along the DNA, the portion of the DNA opened by the RNA polymerase closes down and re-bonds after leaving the RNA polymerase. In this detection method, the RNA polymerase is attached to an electronic device, such as a single electron transistor. Whenever the polymerase replication takes place, a charge variation may occur on the single electron transistor for each portion of the DNA molecule opened up by the RNA polymerase. By detecting these charge variations, the composition of the portion of the DNA molecule that is transcribed can be determined.

Unfortunately, the polymerase chain reaction method relies on the occurrence of this biological process of replication. In addition, the RNA polymerase replication only begins and ends at certain defined points of the DNA strand. As a result, it may be difficult to discover all portions of the DNA strand to be examined.

DNA and RNA can also be sequenced using a chemical method. The chemical sequencing procedure begins by labeling one end of single stranded DNA or RNA with radioactive phosphorous. The labeled strands are then exposed to a mild chemical treatment that is targeted to destroy only one kind of the four different kinds of DNA or RNA subunits. Because the treatment is mild, usually only a single subunit is destroyed in each strand of DNA. This generates a family of fragments of different lengths reflecting the different sites at which the particular destroyed type of subunit occur in the original molecule. These fragments are then separated on a gel and detected using autoradiography to reveal the locations of the radioactive phosphorous. Similar procedures are carried out simultaneously on fresh samples for each of the remaining three polymeric subunits. All four digestions can be separated in individual lanes on a gel and the sequence can be read off in order of size by which polymeric subunit was destroyed.

Unfortunately, this complicated chemical processing method is expensive, cumbersome, and slow. While the process has been automated, there are still definite limits the length of RNA or DNA that can be sequenced. In addition, the use of radioactive labels can make this method of sequencing environmentally damaging over the long term.

In addition to the sequencing of DNA and RNA, polypeptides or proteins can also be sequenced by various methods. One such method is known as N-terminal sequencing. N-terminal sequencing uses the Edman degradation process to cleave the peptide bonds between the amino acids that make up the polypeptide. The peptide bonds are then cleaved, one at a time, starting from the N-terminus of a polypeptide sample. The cleaved amino acids are then analyzed according to the speed at which they flow through a particular column in order to determine which amino acid was cleaved off. The whole process is then repeated for each amino acid in the chain until the whole sequence is determined. Unfortunately, this process requires a substantial amount of purified polypeptide and long processing times. Longer sequences must be sequenced overnight or over days. Furthermore, the sample is destroyed in the process of sequencing.

Another approach to polypeptide sequencing involves C-terminal sequencing. This approach uses a modified Edman process to cleave the peptide bonds between the amino acids, one at a time, starting from the C-terminus. The amino acids are then analyzed, one at a time, in a manner similar to that for N-terminal sequencing. In addition to having the same drawbacks as N-terminal sequencing, C-terminal sequencing is relatively primitive. Generally, sequences of no more than 5-10 amino acids can be obtained. In addition, considerably more starting material is required for C-terminal sequencing than for the N-terminal process.

Devices and methods having the flexibility to examine the entire sequence of a biological polymer, without requiring complicated chemical and optical processing, are needed. A molecule detection system using nanoelectronic devices without the requirement of a biological replication process may be a smaller and less costly system than conventional approaches. This integrated molecule detection system would be easier to use and may be adaptable to detect a variety of predetermined sets of nucleotides or amino acids within a biological polymer. Furthermore, this molecule detection system may be integrated with other electronic devices for further analysis and categorization of the detected molecules.

### BRIEF SUMMARY OF THE INVENTION

The present invention, in a number of embodiments, includes molecular analysis devices and methods for detecting the constituent parts of molecules. A representative embodiment of a molecular analysis device comprises at least one molecule sensor and at least one nanopore. The at least one nanopore is disposed through, partially through, or substantially near the at least one molecule sensor. The at least one molecular sensor may be a single electron transistor or a nanowire.

Another representative embodiment includes a method of detecting a molecule. The method includes guiding at least a portion of the molecule through a nanopore that passes through, partially through, or substantially near a molecular sensor. The method further includes sensing an electronic effect responsive to the molecule passing through, partially through, or substantially near the molecule sensor. The molecule sensor may be a single electron transistor or a nanowire.

### BRIEF DESCRIPTION OF THE SEVERAL VIEWS OF THE DRAWINGS

While the specification concludes with claims particularly pointing out and distinctly claiming that which is regarded as the present invention, the advantages of this invention can be more readily ascertained from the following description of the invention when read in conjunction with the accompanying drawings in which:

FIG. 1 is a three dimensional view of a portion of a DNA molecule;

FIG. 2 is a flat view of a portion of a DNA molecule showing various possible base pair bondings;

FIG. 3 illustrates the chemical structure of a short representative polypeptide molecule;

FIG. 4 is a table of the 20 most common amino acids that make up polypeptides and their abbreviations;

FIG. 5 is a top view of an exemplary molecular analysis device including a nanopore and a molecule sensor;

FIGS. 6A and 6B are exemplary cut-away views of FIG. 5 illustrating exemplary locations of a nanopore in relation to a molecule sensor;

FIGS. 7A and 7B are three dimensional views of exemplary configurations of a nanopore and a molecule sensor;

FIG. 8 is a top view of another exemplary molecular analysis device including a nanopore and a molecule sensor;

FIG. 9A is a cut-away view of FIG. 8 illustrating the location of a nanopore in relation to a molecule sensor;

FIG. 9B is a three dimensional view of the nanopore and molecule sensor of FIG. 8;

FIG. 10 is a top view of an exemplary molecular analysis device including a plurality of nanopores and a plurality of molecule sensors;

FIG. 11 is a three dimensional view of an embodiment of a molecule sensor comprising a single electron transistor;

FIG. 12 is a schematic view of an exemplary single electron transistor;

FIG. 13 is a graphical view of an electrical characteristic of an exemplary single electron transistor;

FIG. 14A is a top view of an exemplary single electron transistor including control electrodes;

FIG. 14B is a scanning electron microscope picture of the exemplary single electron transistor of FIG. 14A;

FIG. 15A is a top view of an exemplary molecular analysis device including a nanopore and a molecule sensor comprising a single electron transistor;

FIG. 15B is a cut-away view of FIG. 15A further illustrating the location of the nanopore in relation to a molecule sensor comprising a single electron transistor;

FIG. 16A is a top view of an exemplary molecular analysis device including a nanopore and a molecule sensor comprising a single electron transistor;

FIG. 16B is a cut-away view of FIG. 16A further illustrating the location of the nanopore in relation to the molecule sensor comprising the single electron transistor;

FIG. 17A is a top view of another exemplary molecular analysis device including a nanopore and a molecule sensor comprising a single electron transistor;

FIG. 17B is a cut-away view of the molecular analysis device of FIG. 17A;

FIG. 18 is a graphical view illustrating the electronic effect on an exemplary single electron transistor sensing a polypeptide

FIG. 19 is a top view of an exemplary single electron transistor including a nitrogenous material disposed on a quantum dot and an exemplary bonding to a nucleic acid chain;

FIG. 20 is a top view of an exemplary single electron transistor including an oligonucleotide disposed on a quantum dot and an exemplary bonding to a nucleic acid chain;

FIGS. 21A, 21B, and 21C are pictorial top views of various embodiments of single electron transistors including various numbers of quantum dots;

FIG. 22 is a top view of a plurality of exemplary nanowires;

FIGS. 23A, 23B, and 23C, are top views illustrating exemplary positions of a nanopore in relation to a nanowire;

FIG. 24 is a top view of an exemplary nanowire including a nitrogenous material disposed on the nanowire and an exemplary bonding to a nucleic acid chain;

FIG. 25 is a top view of an exemplary nanowire including an oligonucleotide disposed on the nanowire and bonding to a nucleic acid chain;

FIG. 26A is a graphical view illustrating a lack of a conductance change in a nanowire with no bonding event;

FIG. 26B is a graphical view illustrating a conductance increase in an exemplary p-type nanowire when a bonding event occurs; and

FIG. 26C is a graphical view illustrating a conductance decrease in an exemplary n-type nanowire when a bonding event occurs.

### DETAILED DESCRIPTION OF THE INVENTION

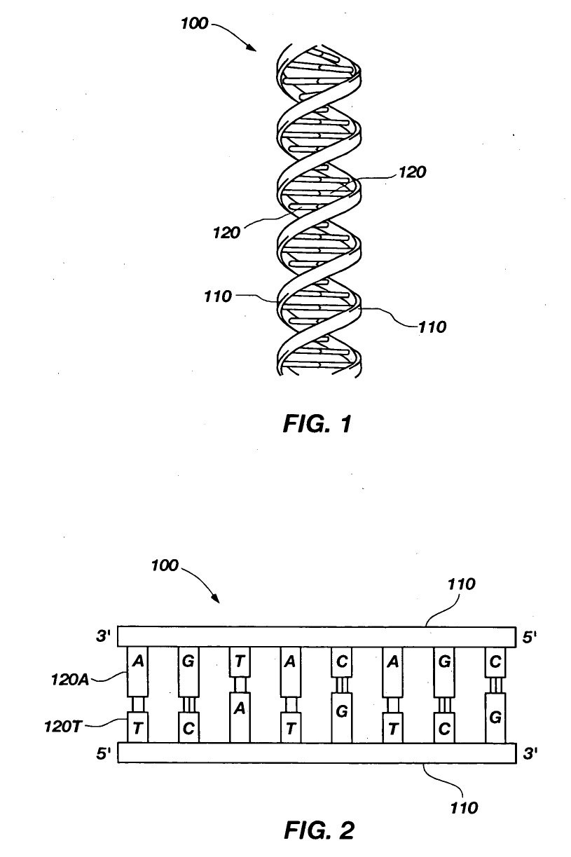

The present invention, in a number of embodiments, includes structures, devices, and methods for use in detecting the molecular structure of biological polymers. As illustrated in FIGS. 1 and 2, an example of one such biological polymer is deoxyribonucleic acid (DNA). A DNA molecule 100 comprises a double helix structure including two backbone strands 110 on the outside of the double helix. The backbone strands 110 are a structure made up of sugar-phosphate polymer strands. Between the two backbone strands 110 are pairs of bases 120 configured similar to ladder rungs. The bases 120 connecting the strands consist of four types: adenine 120A (A), thymine 120T (T), guanine 120G (G), and cytosine 120C (C). RNA, which is closely related to DNA, comprises a similar structure including the A, G, and C bases of DNA. However, in RNA, instead of bonding with T, A bonds with the molecule uracil (U) (not shown), which is closely related to T.

Each of the base molecules 120 comprise nitrogenous compounds in various configurations. The base molecules 120 may bond with each other to form base pairs. As shown in FIG. 2, T may form two hydrogen bonds with A, while C may form three hydrogen bonds with G. These hydrogen bonds between the base pairs are relatively weak, allowing a DNA strand to be separated into two complementary single stranded molecules. A single human DNA molecule may include as many as three billion of these base pairs.

Another way of characterizing the constituent parts of a DNA strand is to consider the various bases 120 chemically bonded to a sugar. In this form, the resultant molecule is often referred to as a nucleoside. Each nucleoside includes a sugar molecule bonded to one of the various bases 120. A nucleoside with a phosphate molecule bonded to the sugar portion of the nucleoside is often referred to as a nucleotide. Thus, each strand of a DNA molecule may be considered as a plurality of nucleotides bonded together, wherein the bonds form at the sugar-phosphate portion of each nucleotide to form the backbone 110 of the strand. Nucleotides join together to form the backbone strands 110 by a 5′-3′ phosphodiester linkage, giving the strands a directionality. Thus, the 5′ end of the strand has a free phosphate group and the 3′ end has a free hydroxyl group. In double stranded DNA, the backbone strands 110 run in opposite directions such that each end of the double strand has a 5′ end on one backbone strand 110 and a 3′ end on the other backbone strand 110.

A section of single stranded DNA including a small plurality of nucleotides is often referred to as an oligonucleotide. These oligonucleotides are conventionally used as the tags in the prior art DNA micro-arrays previously described.

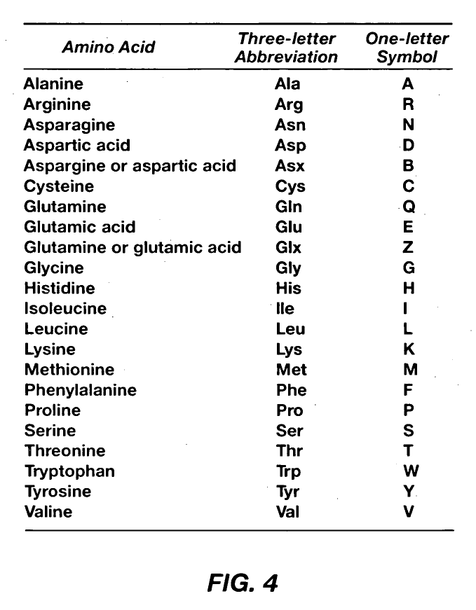

In genetic coding, an oligonucleotide comprising three consecutive nucleotides along RNA or single stranded DNA is often referred to as a codon. Any three consecutive nucleotides of A, C, G, and T (or U for RNA), can be combined in 64 (i.e., 4³) possible combinations. 20 different amino acids (see FIG. 4) are specified by these 64 different codons and are represented by one or more codons. For example, the amino acid Alanine may be represented by the codons GCA, GCC, GCG, and GCU.

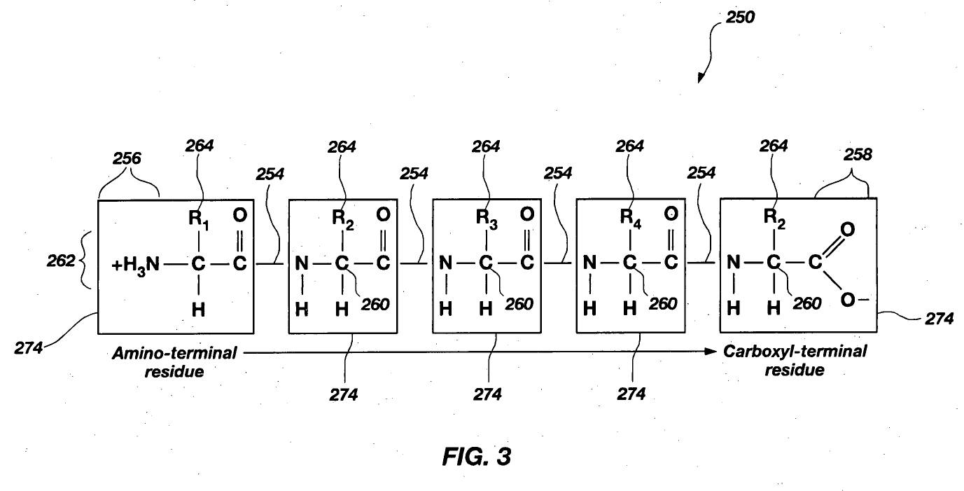

Another example of such a biological polymer is a polypeptide or protein. Referring now to FIG. 3, there is shown a representative polypeptide molecule 250, which may comprise a series of two or more amino acids 274 joined together by peptide bond 254. Polypeptides have an amino- or N-terminus 256 and a carboxy- or C-terminus 258. The peptide bonds and the intervening α-carbons 260 make up the backbone of the polypeptide, generally at 262, while the side chains 264 (depicted herein as R₁—R₅) vary among the individual amino acids. The twenty most common amino acids and their abbreviations are provided in FIG. 4. These 20 amino acids make up more than 99% of all the amino acids found in proteins.

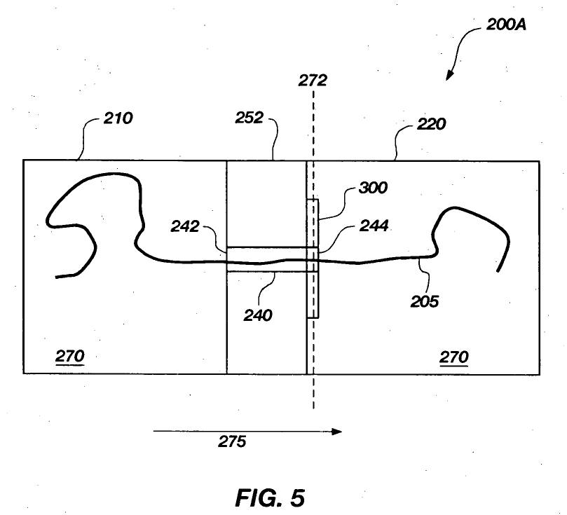

FIG. 5 illustrates one of many possible configurations of a representative embodiment of a molecular analysis device 200A for analyzing biological polymers such as nucleic acid chains or polypeptides. The molecular analysis device 200A includes a supply reservoir 210, an accumulation reservoir 220, a molecule guide (also referred to as a nanopore 240 which is shown disposed through a membrane 252 in the embodiment of FIG. 5), and a molecule sensor 300. In addition, a transport medium 270, such as, for example, an electrolyte solution, may be contained within the supply reservoir 210, the nanopore 240, and the accumulation reservoir 220. At least one biological polymer chain 205 may be disposed within the transport medium 270. The molecule sensor 300 is described in more detail below.

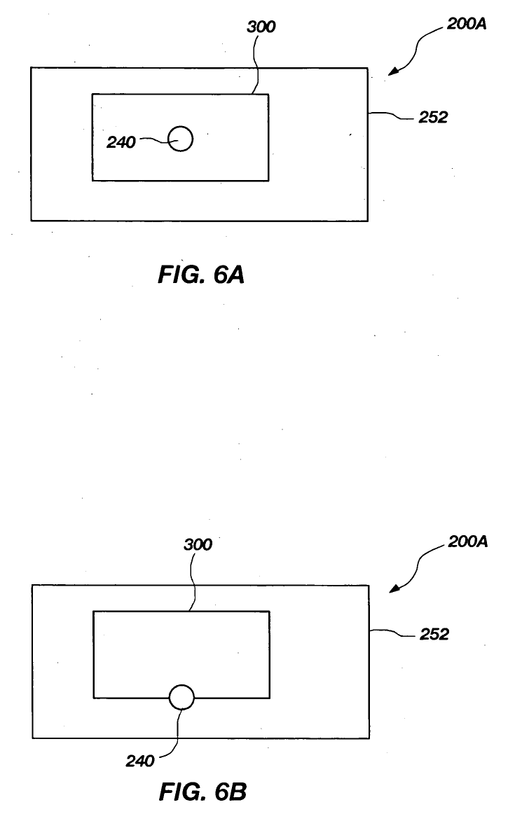

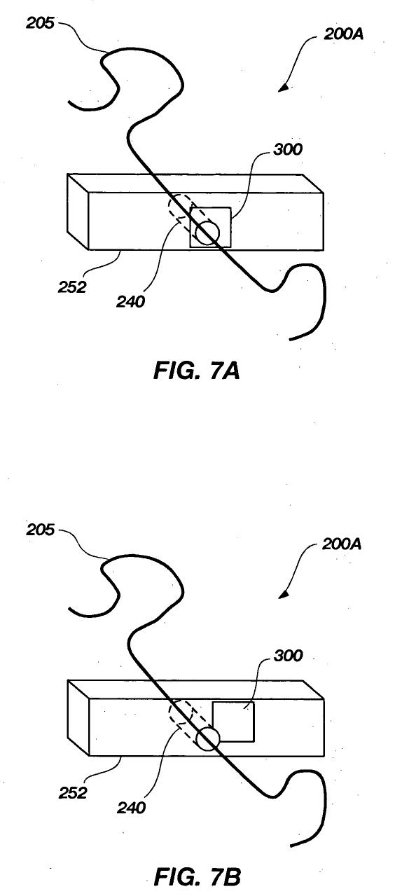

Referring now to FIGS. 6A and 6B, there are shown representative cut-away views of device 200A along plane 272. These cut-away views more clearly illustrate possible positions of the nanopore 240 in relation to sensor 300. Referring now to FIGS. 7A and 7B, there are shown three dimensional views that more clearly illustrate possible positions of the nanopore 240 in relation to sensor 300. As will be apparent to one of skill in the art, any configuration of a nanopore 240 and a sensor 300 in which the nanopore 240 passes wholly or partially through sensor 300 is contemplated as being within the scope of device 200A.

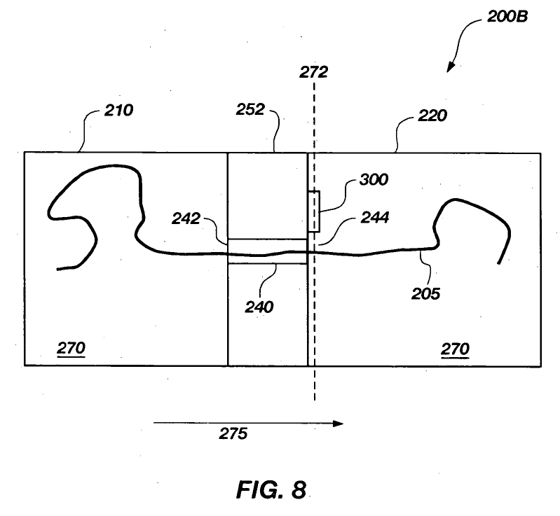

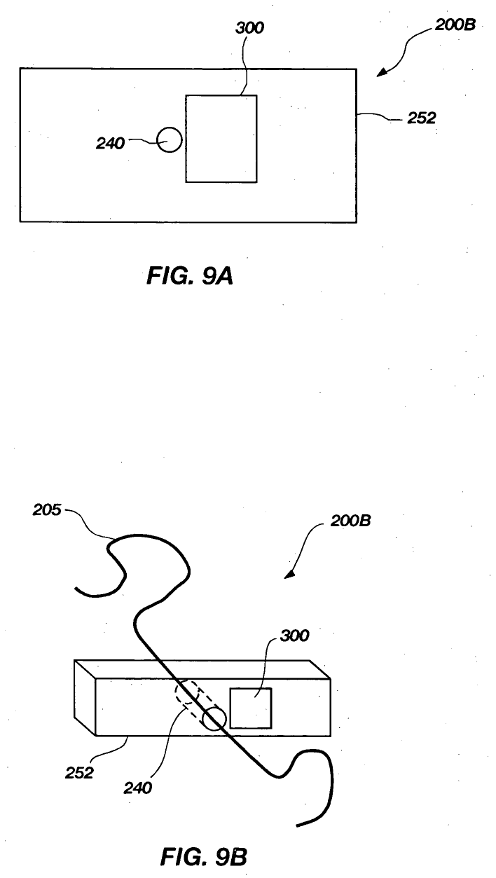

Referring now to FIGS. 8, 9A, and 9B, there is illustrated one of many further possible configurations of a representative embodiment of a molecular analysis device 200B for analyzing biological polymers. As shown more clearly in FIGS. 9A and 9B, nanopore 240 may be located substantially near sensor 300. As will be apparent to one of skill in the art, any configuration of a nanopore 240 and a sensor 300 in which the nanopore 240 is disposed substantially near sensor 300 is contemplated as being within the scope of device 200B.

The nanopore 240 may be configured for carrying the biological polymer chain 205 in the transport medium 270 from the supply reservoir 210, through the nanopore 240, to the accumulation reservoir 220 in the transport direction 275 shown. Alternatively, the transport medium 270 may be configured for carrying the biological polymer chain 205 from the accumulation reservoir 220, through the nanopore 240, to the supply reservoir 210. Various methods may be used to transport the biological polymer chain 205 through the nanopore 240, such as, by way of example, electrokinetic flow, electroosmotic flow, hydrostatic pressure, hydrodynamic pressure, and hydromagnetic flow. These transport mechanisms may be caused mechanically, magnetically, with an electrical field, by heat-induction, or any other methods known to a person of ordinary skill in the art.

Electrophoresis causes the movement of particles that are suspended in a medium to which an electromotive force is applied. Particularly, a particle or molecule having an electrical charge will experience an electromotive force when positioned within an electrical field. Nucleic acid chains such as DNA molecule 100 are good candidates for electrophoresis because they carry multiple negative charges due to the phosphodiester backbone 110 (FIGS. 1 and 2). Polypeptides can be easily made to carry a net negative charge by placing them in the presence of sodium dodecyl sulfate (SDS). Thus, when electrodes (not shown) with a voltage differential are placed in the transport medium 270, the biological polymer chains 205 will migrate toward the more positive electrode. By way of example, if an electrode with a ground potential is placed in the supply reservoir 210 and an electrode with a positive voltage is placed in the accumulation reservoir 220, then biological polymer chains 205 in the transport medium 270 can migrate from the supply reservoir 210, through the nanopore 240, and toward the electrode in the accumulation reservoir 220. Furthermore, the movement rate or velocity of the biological polymer chain 205 substantially correlates with the voltage bias between the electrodes. As a result, a first approximation of the biological polymer chain 205 velocity may be determined, which may be used by, and refined by, signal processing analysis in combination with signal data from the molecule sensor 300 to determine the constituent parts of the biological polymer chain 205.

Other transport mechanisms may rely on nanofluidic flow of the transport medium 270 itself, with the biological polymer chain 205 being carried along with the transport medium 270. For example, electrokinetic flow (often referred to as electroosmotic flow) is generated in a manner similar to electrophoresis by electrodes (not shown) in the supply reservoir 210 and the accumulation reservoir 220. Electrokinetic flow of the transport medium 270 may generally require higher voltage potentials to cause transport medium 270 flow than the voltage required to cause electrophoretic movement of the biological polymer chains 205. Thus, biological polymer chain 205 movement may be substantially electrophoretic or may be a combination of electrophoretic movement and movement caused by electrokinetic flow of the transport medium 270.

Yet another transport mechanism may rely on pressure driven flow. In very small channels or openings, such as nanopores 240, a small pressure differential may be developed by applying a temperature differential between the supply reservoir 210 and the accumulation reservoir 220. This small pressure differential may cause the flow of the transport medium 270, and biological polymer chains 100 within the transport medium 270, from one reservoir (210, 220) to the other reservoir (220, 210).

A nanopore 240, as shown in FIGS. 5 through 9B, has an opening of from about 1 nanometer to about 100 nanometers and is disposed through a membrane 252. The membrane 252 may comprise an organic or inorganic material, which may be fabricated using a variety of lithographic techniques, nano-imprint lithographic techniques, self-assembly techniques, or combinations thereof.

The nanopore 240 may be cylindrical in shape (as shown in FIGS. 5 through 9B) or may include other cross sectional shapes, such as, by way of example, triangular, square, hexagonal, and octagonal. The figures illustrating nanopores 240 in membranes 252 are generally shown with a nanopore 240 configured horizontally through a vertical membrane 252. However, the membrane 252 may be disposed horizontally, with a vertical nanopore 240 therethrough, or any other suitable configuration, so long as the nanopore 240 may be configured to pass successive segments of the biological polymer chain 205 through, partially through, or substantially near the molecule sensor 300, as explained below.

In a particular embodiment, the nanopore 240 may be about 100 nm or less to ensure the biological polymer chain 205 does not pass through the nanopore 240 in some type of looped configuration. To ensure that the biological polymer chain 205 is presented through, partially through, or substantially near the molecule sensor 300, the nanopore 240 may need to be significantly narrower than the width needed to keep the biological polymer chain 205 from forming loops. Thus, the cross sectional dimensions of nanopore 240 may vary depending on the type of molecule sensor 300 used, as well the type of biological polymer chain 205 to be sensed.

The membrane 252 may have a wide variety of thicknesses because the invention uses the nanopore 240 as a presentation and transport mechanism, rather than a sensing mechanism. A relatively thin membrane 252 may enable more uniform nanopores 240. A relatively thick membrane 252 may assist in straightening the biological polymer chain 205 in the vicinities of the nanopore 240, entrance point 242, and nanopore 240 exit point 244.

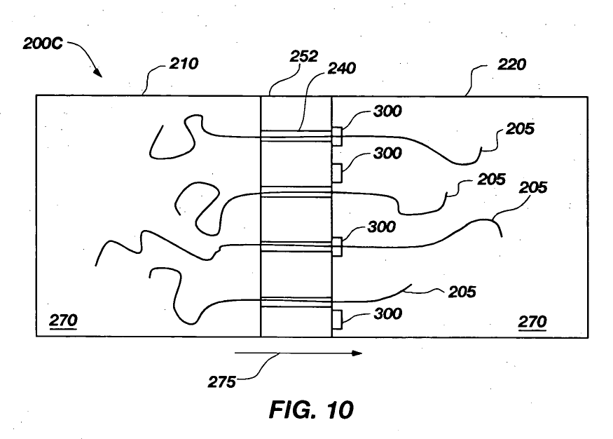

FIG. 10 illustrates an embodiment of a molecular analysis device including a plurality of nanopores and a plurality of molecule sensors. The plurality of nanopores 240 are coupled to a single supply reservoir 210 and a single accumulation reservoir 220, and are adapted to receive a biological polymer chain 205 in each of the plurality of nanopores 240 in a transport direction 275 from the supply reservoir 210 to the accumulation reservoir 220. A person of ordinary skill in the art will appreciate that many configurations of reservoirs (210, 220), nanopores 240, and molecule sensors 300 are contemplated as being within the scope of the invention.

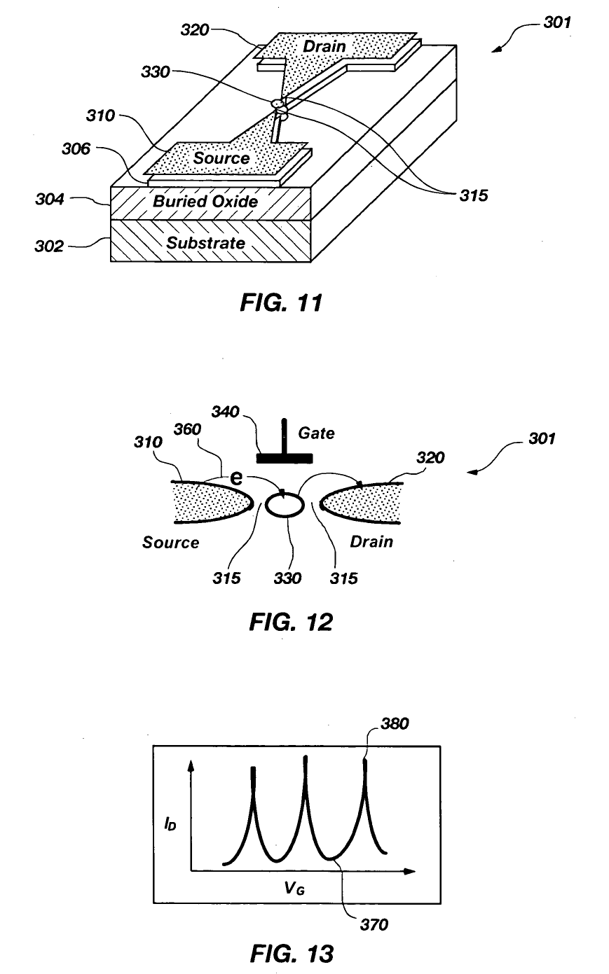

FIG. 11 illustrates a representative molecule sensor 301 configured as a single electron transistor (SET). The SET 301 includes a source 310 (also referred to as a first terminal) and a drain 320 (also referred to as a second terminal). A quantum dot 330, positioned between the source 310 and drain 320, is embedded in a tunneling layer 306. Suitable tunneling layers include silicon dioxide or any other suitable dielectric. The dielectric forms tunneling junctions 315. One tunneling junction 315 operably couples the source 310 to the quantum dot 330, and another tunneling junction 315 operably couples the drain 320 to the quantum dot 330. The representative molecule sensor 300 may be formed on a silicon substrate 302 with a buried oxide layer 304 formed thereon.

A SET operates similarly to a field effect transistor (FET), except that in a conventional conducting FET, thousands or millions of electrons may traverse from the source 310 to the drain 320. In a SET 301, as few as one electron at a time may leave the source 3 10 node or arrive at the drain 320 node.

A SET 301 may include two primary phenomena: a single electron effect and a quantum effect. Until the feature sizes of the SET 301 become extremely small (e.g., less than 5 nm for a quantum dot 330 embedded in SiO₂), the single electron effect dominates. In understanding the single electron effect, the quantum dot 330 may be considered like a capacitor. The electrostatic energy stored in a capacitor with a charge of q is given by:

$$E = \frac{{q}^{2}}{2 C}$$

If the capacitance is small enough, the electrostatic energy of one electron may be larger than the thermal energy, as represented by:

$$\frac{{e}^{2}}{2 C} \geq {k}_{B} T$$

where ‘e’ represents the charge of one electron and ‘k_{b}’ represent the Boltzman constant. If the electrostatic energy of one electron is larger than the thermal energy, the energy stored in the capacitor does not change continuously, and the charge and discharge of one electron onto the capacitor leads to an observable change in total energy.

For example, assume there are n electrons stored in the capacitor and one more electron (i.e. an n+1 electron) is to be charged onto the capacitor. The total electrostatic energy of the capacitor before the n+1 electron is charged is:

$${E}_{n} = \frac{{n}^{2} {e}^{2}}{2 C}$$

Similarly, the total electrostatic energy of the capacitor after the n+1 electron is charged is:

$${E}_{n + 1} = \frac{{( n + 1 )}^{2} {e}^{2}}{2 C}$$

Therefore, the energy needed to charge the N+1 electron is:

$$Δ E = {E}_{n + 1} - {E}_{n} = \frac{{( n + 1 )}^{2} {e}^{2}}{2 C} - \frac{{n}^{2} {e}^{2}}{2 C} = ( n + 1 / 2 ) \frac{{e}^{2}}{C}$$

The electrostatic energy levels in the capacitor comprise discrete energy levels, where the lowest energy level is cE₀=e²/2C and the energy between each subsequent level is described as ΔcE′=e²/C.

As noted, to observe these single-electron effects, the energy spacing between each discrete energy level must be larger than the thermal energy. For example, for a quantum dot 330 embedded in SiO₂, the quantum dot 330 will typically have a diameter of about 10 nm or less for the energy level spacing to be about three times larger than the thermal energy at room temperature.

If the quantum dot 330 is small enough to make the gap between each energy level larger than the thermal energy, then the energy inside the dot has a discrete spectrum. Tunneling of electrons from the source 310 to the quantum dot 330 or from the quantum dot 330 to the drains 320, via the tunneling junctions 315, is inhibited until the energy gap is overcome through an applied bias between the source 310 and drain 320. In other words, electrons only transfer from the source 310 to the quantum dot 330, one by one. This phenomenon is known as a Coulomb blockade.

Clear Coulomb blockade effects may be observed when the tunneling resistance between the quantum dot 330 and other terminals is larger than about 26 kOhms. This tunneling resistance at which Coulomb blockade effects are seen is often referred to as the “quantum resistance.”

When the energy levels of the source 310 and drain 320 misalign with the energy level of the quantum dot 330, the SET 301 exhibits low conductance between source 310 and drain 320, inhibiting electron transfer. Conversely, when the energy levels of the source 310 and drain 320 align with the energy level of the quantum dot 330, the SET 301 exhibits high conductance, enabling electron transfer.

A gate electrode 340, as shown in FIG. 12, may be placed close enough to the quantum dot 330 to affect the amount of energy needed to change the number of electrons on the quantum dot 330. For example, assuming the bias voltage between the source 310 and drain 320 is held at a level below the coulomb blockade voltage, as voltage on the gate 340 is increased, the energy level on the quantum dot 330 near the tunneling junctions 315 changes. At a certain point, the energy level of the source 310 and drain 320 will align with the energy level of the quantum dot 330 near the tunneling junction 315 and a new electron may be added to the quantum dot 330. When the electron is added, the SET 301 returns to a Coulomb blockade because the new energy level of the quantum dot 330 no longer aligns with the energy level of the source 310 and drain 320. Thus, for more electrons to move, the bias between the source 310 and drain 320 must change, or the gate 340 voltage must change, to overcome the Coulomb blockade. This makes the SET 301 very sensitive to charge changes on the gate 340 or other charges substantially near the quantum dot 330.

FIG. 13 illustrates the Coulomb blockade effect as a gate voltage versus drain current at a fixed source to drain bias level. The gate voltage is shown on the x-axis and the drain current is shown on the y-axis. As explained earlier, as the gate voltage increases, the SET 301 will reach a high conductance state 380, enabling electrons to transfer. However, a further increase will place the SET 301 in a low conductance state 370 inhibiting electron transfers 360.

One reason a SET 301 is useful for analysis of biological polymer chains 205 is because of the charge sensitivity of a SET 301. A charge does not need to be in the quantum dot 330, it just needs to be close enough to influence the energy level of the quantum dot 330. This is often referred to as the Debye length, which is usually about 17 nm for lightly doped silicon. Thus, when a charged molecule is within the Debye length, the SET 301 will be able to detect the charge.

The Debye length can also help with noise rejection because the SET 301 is not influenced by a charge located farther away than the Debye length. However, the Debye length also means that the nanopore 240, adjustment electrodes 340 (shown in FIGS. 14A and 14B, and explained below), or combinations thereof, must bring the biological polymer chain 205 close enough to the quantum dot 330 to sense the intrinsic charge of the biological polymer chain 205 at the location substantially near the quantum dot 330.

FIG. 14A illustrates a representative SET 30f, including the source 310, drain 320, and quantum dot 330. FIG. 14B is a scanning electron microscope picture (rotated 90 degrees counter-clockwise) of the representative single electron transistor of FIG. 14A. The FIG. 14A embodiment of the SET 301 also includes two electrodes 340 near the quantum dot 330. The electrodes 340 may be used as gates to the SET 301 to influence the Coulomb blockade level. The electrodes 340 may also perform an additional function. Because a biological polymer chain 205 is negatively charged, the voltage of the electrodes 340 may be adjusted to cause the biological polymer chain 205 to move forward or backward relative to the quantum dot 330. This may be thought of as a way to “fine-tune” the movement of the biological polymer chain 205, which is caused by the electrophoresis or other transport mechanism described above. This fine-tuning also may be used to achieve a better alignment of the biological polymer chain 205 relative to the quantum dot 330.

While not shown in the figures, another embodiment of the SET 301 may include a single electrode 340. However, two electrodes 340, one on each side of the quantum dot 330, may give additional control, enabling controllable movement of the biological polymer chain 205 in both directions relative to the quantum dot 330. In yet another embodiment of the SET 301 (not shown), the gate 340 may be formed over the quantum dot 330 creating a gap between the quantum dot 330 and the gate 340, through which the transport medium 270 and the biological polymer chain 205 may pass.

In addition, the discussion has focused on a silicon quantum dot implementation of a SET. However, other SET implementations are contemplated as being within the scope of the invention. For example, SETs may be formed using metal as the quantum dot. Typically, these SETs use an aluminum quantum dot, with aluminum oxide to form the tunneling junctions. As another example, SETs may be formed on III-V materials, such as GaAs, using metal gates as the quantum dot. These SETs would usually have application at low temperatures due to the large quantum dot size, which requires a low thermal energy.

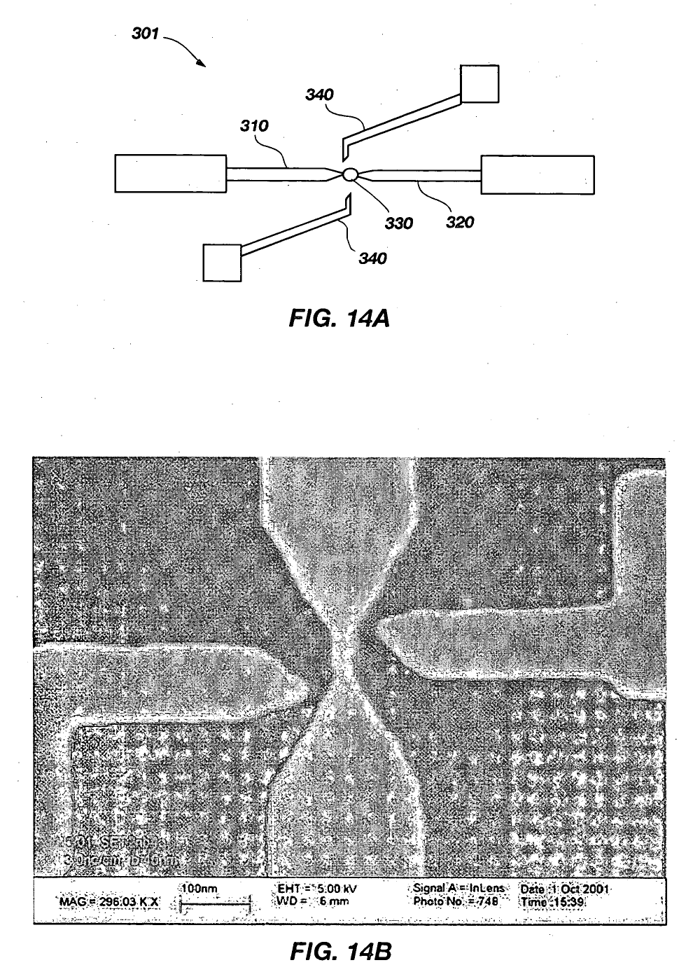

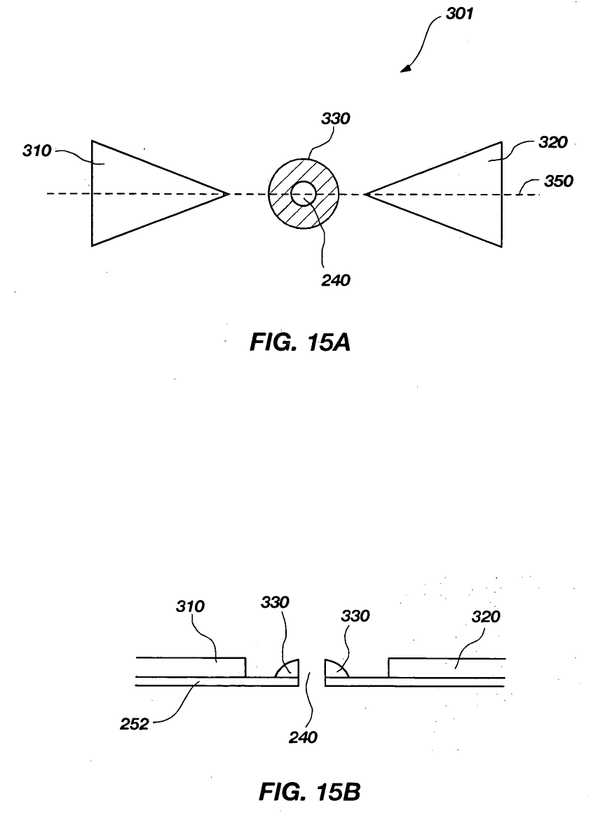

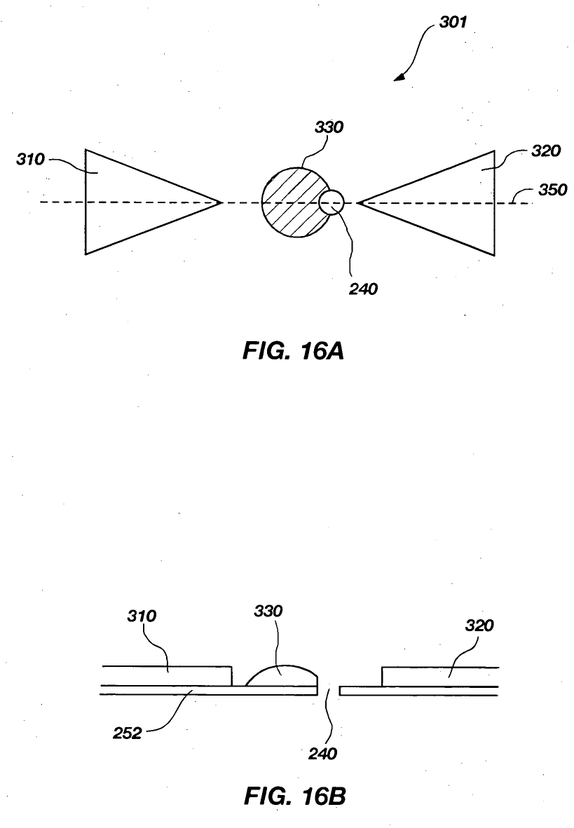

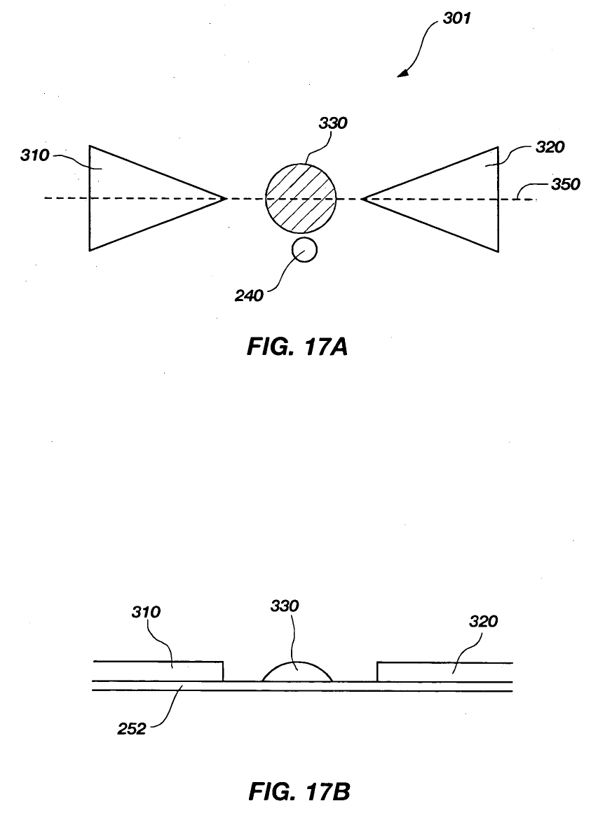

FIGS. 15A through 17B illustrate representative configurations of a nanopore 240, source 310, drain 320, and quantum dot 330 of a representative SET 301. Although not illustrated, membrane 252 may encompass a substrate 302 and/or a buried oxide layer 304, and may further include tunneling layer 306. As shown in FIG. 15A, the nanopore 240 may pass completely through the quantum dot 330. This can be more clearly seen in the cut-away of FIG. 15A along line 350 as shown in FIG. 15B. As shown in FIG. 16A, the nanopore 240 may-pass partly through the quantum dot 330. This can again be more clearly seen in the cut-away of FIG. 16A along line 350 as shown in FIG. 16B. Lastly, as shown in FIG. 17A, the nanopore 240 may pass substantially near the quantum dot 330. FIG. 17B again provides a cut-away of 17A along line 350. As will be apparent to one of skill in the art, any configuration of a nanopore 240 and a quantum dot 330, in which nanopore 240 passes wholly through, partially through, or substantially near to quantum dot 330 is, contemplated as being within the scope of the invention.

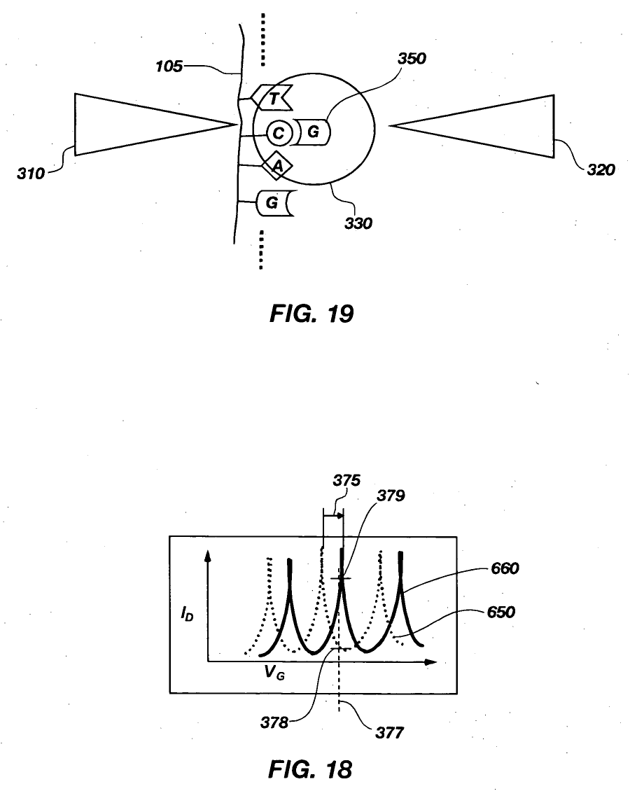

In operation, biological polymer chain 205 may comprise for example, a polypeptide. As the individual amino acids pass through a nanopore 240 and therefore through, partially through, or substantially near quantum dot 330, there will be an electronic effect 375 (shown in FIG. 18) in the SET 301 due to the charge difference near the quantum dot 330.

FIG. 18 illustrates this electronic effect 375 as a gate voltage versus drain current at a fixed source to drain bias level. A first curve 650 (shown as a dotted line) illustrates the SET 301 characteristics before the amino acid was close enough to influence quantum dot 330. A second curve 660 (shown as a solid line) illustrates a shift in the characteristics of the SET 301 due to the change in charge near the quantum dot 330. If a gate bias is set at a sampling level where the SET 301 is in a relatively low conductance state 378, then the shift in characteristics due to the change in charge near the quantum dot 330 may cause the SET 301 to move to a higher conductance state 379. This higher conductance at the drain 320 may be sensed by other electronic devices on the substrate 302 to give an indication that a particular type of amino acid was being sensed by the quantum dot. For example, an arginine amino acid was, at that time, passing through, passing partially through, or substantially near the quantum dot 330. It will be apparent to one of skill in the art that, while FIG. 18 describes the effect of an amino acid in a polynucleotide, any biological polymer may be used with similar results.

Signal processing hardware, software, or combination thereof, may then be used to gather and process data of the times when amino acids are substantially near the quantum dot 330 and the speed of the polynucleotide chain.

The quantum dot 330 in a representative SET 301 may also be coated with a nitrogenous material 350. For example, for detecting portions of a polynucleotide chain 105 (such as DNA or RNA), the nitrogenous material 350 may comprise a base selected from the group consisting of adenine 120A, thymine 120T, uracil 120U, cytosine 120C, and guanine 120G. Furthermore, the nitrogenous material 350 coating the quantum dot 330 may also include a sugar bonded to the base or a sugar-phosphate bonded to the base. By way of example, FIG. 19 illustrates the nitrogenous material 350 guanine (120G of FIG. 2). FIG. 19 illustrates a representative symbol for the guanine 120G to show functional interaction with a polynucleotide chain 105. However, generally, the entire quantum dot 330 may be coated with the nitrogenous material 350.

As the polynucleotide chain 105 passes through a nanopore 240 and therefore through, partially through, or substantially near the coated quantum dot 330, a base 120 (in this example, C) of the polynucleotide chain 105 that is complementary to the nitrogenous material 350 (in this example, G) on the quantum dot 330 may react with the nitrogenous material 350. This reaction may take the form of a transitory chemical bond between the complementary base on the polynucleotide chain 105 and the nitrogenous material 350 on the quantum dot 330. The transitory chemical bond will cause an electronic effect 375 (similar to the effect shown in FIG. 18) in the SET 301, due to the charge difference near the quantum dot 330.

As with the polypeptides sensed in FIG. 18, the transitory chemical bond between the polynucleotide chain 105 and the attached nitrogenous material 350 will cause an electronic effect in the SET 301 due to the charge difference near the quantum dot 330. This electronic effect 375 will be similar to that shown in FIG. 18, but perhaps with a different magnitude than that shown for the SET 301 that is not coated with a nitrogenous material 350. A plurality of molecule sensors 300 configured with a variety of nitrogenous materials 350 may be useful in determining different specific characteristics of any given polynucleotide chain 105.

Signal processing hardware, software, or combination thereof, may then be used to gather and process data of the times when individual bases of polynucleotide chain 105 are substantially near the quantum dot 330 and the speed of the polynucleotide chain 105. If other molecule sensors 300 which are sensitive to the other bases 120 (i.e., A, T, G, and U) are configured in the nanochannel 240, a complete solution of the polynucleotide chain 105 may be derived based on the velocity of the polynucleotide chain 105 and the relative positioning of the various molecule sensors 300.

In addition, a polynucleotide molecule is negatively charged and the magnitude of the charge is proportional to the length of the molecule. Thus, because the SET 301 is sensitive to charge variations, the SET 301 may also be used to determine the molecules overall length and the current position of the molecule relative to the SET 301.

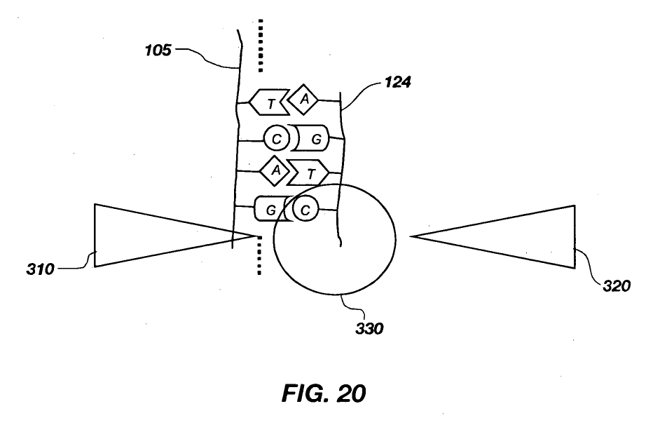

FIG. 20 illustrates the source 310, drain 320, and quantum dot 330 of another representative SET 301. The quantum dot 330 in this representative SET 301 includes an oligonucleotide 124 attached to the quantum dot 330. The oligonucleotide 124 may include many combinations of nucleotides and may have various lengths to comprise a specific combination of nucleotides that may be of interest. By way of example, FIG. 20 illustrates an oligonucleotide 124 including four nucleotides in the series of C, T, G, and A.

The attachment of the oligonucleotide 124 to the SET 301 may be accomplished with a variety of methods know to those of ordinary skill in the art, such as the methods used in micro-arrays.

As the polynucleotide chain 105 passes through the nanopores 240 and, therefore, substantially near the attached oligonucleotide 124, if a complementary sequence of bases passes substantially near the attached oligonucleotide 124, a transitory chemical bond (i.e., hybridization) may occur between the oligonucleotide 124 and the complementary sequence on the polynucleotide chain 105. In the representative embodiment of FIG. 20, the oligonucleotide 124 comprising the sequence C, T, G, A, may hybridize with the complementary sequence G, A, C, T on the polynucleotide chain 105. As with the single base example of FIG. 19, this transitory chemical bond between the polynucleotide chain 105 and the attached oligonucleotide 124 will cause an electronic effect in the SET 301, due to the charge difference near the quantum dot 330. This electronic effect will be similar to that seen in the embodiment of FIG. 10, but perhaps with a different magnitude than that seen in the SET 301 coated with a nitrogenous material 350. A plurality of molecule sensors 300 configured with a variety of oligonucleotides 124 may be useful in determining different specific characteristics of any given polynucleotide chain 105.

The transitory chemical bond results from weak hydrogen bonds between the oligonucleotide 124 on the quantum dot 330 and the polynucleotide chain 105. The transitory chemical bond may be broken, allowing continued transportation of the polynucleotide chain 105 by the motive force (e.g., thermal energy, optical energy, or combinations thereof) causing transportation of the polynucleotide chain 105.

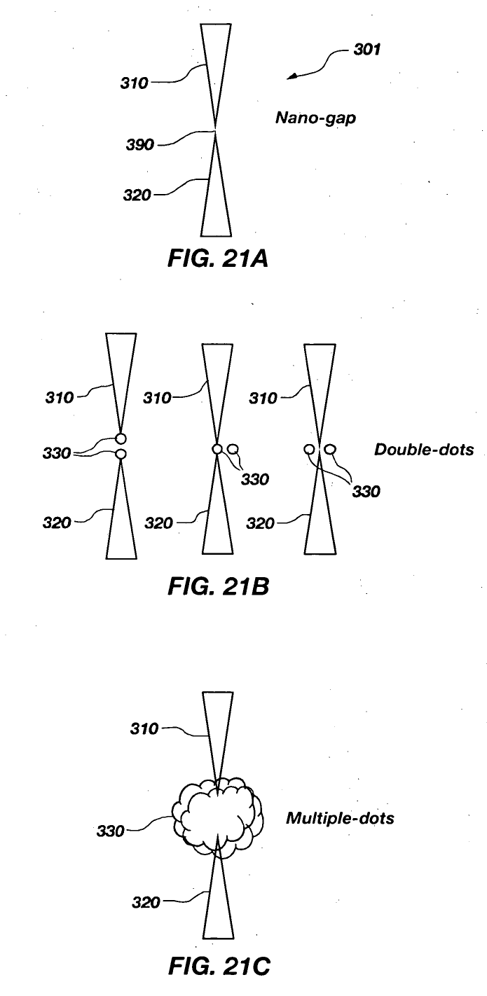

FIGS. 21A, 21B, and 21C illustrate other embodiments of the molecule sensors 300 according to the invention. In some cases, it may be desirable to include two or more quantum dots 330 between the source 310 and drain 320, as illustrated in FIGS. 21B and 21C. Although not illustrated, it will be appreciated that nanopores may be disposed through, partially through, and/or substantially near any or all of the quantum dots illustrated in FIGS. 21B and 21C. The presence of multiple quantum dots may increase sensitivity, noise immunity, or combinations thereof. The operation of multiple dot SETs is similar to that described for the single dot SET 301, except that it may be possible to shift the sensing voltage of different quantum dots 330 based on their location relative to gate electrodes. This may generate more sensitivity to shifts in the SET characteristics to a charge substantially near the quantum dots 330.

FIG. 21A illustrates a representative nanogap implementation of a molecule sensor with no quantum dot and only a single tunneling junction in the gap between the source 310 and drain 320. In the nanogap 390 embodiment, the nitrogenous material 350 or oligonucleotide 124 is disposed at the nanogap 390. With this configuration, the charge difference, due to the presence of a molecule or a transitory chemical bond substantially near the nanogap 390, may cause a difference in the tunneling characteristics of the nanogap 390 and, as a result, the current flowing between the source 310 and drain 320. As described for other SETs, a nanopore 240 may be disposed through, partially through, or substantially near nanogap 390.

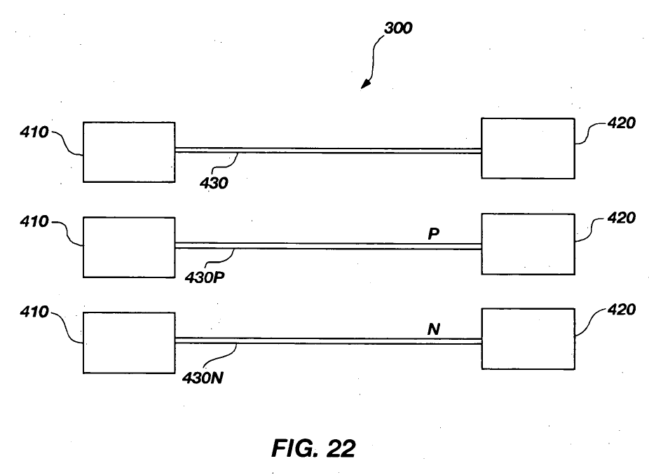

FIG. 22 illustrates another embodiment of molecule sensors 300 configured as nanowires 430. Each nanowire 430 is disposed on a substrate (not shown) between a first terminal 410 and a second terminal 420. These terminals (410, 420) may be used to couple to an apparatus for sensing a conductance change, couple to other semiconductor circuitry on the substrate for sensing a conductance change in the nanowire 430, or combinations thereof, as explained below.

The representative nanowires 430 may be fabricated as silicon nanowires 430 on a silicon substrate with an insulating silicon dioxide layer. However, other substrates suitable for bearing and fabricating semiconductive nanowires 430 are contemplated as being within the scope of the present invention. In addition, the representative nanowires 430 may be doped by ion implantation using a doping material, such as, for example boron and phosphorous to create p-type doping and an n-type doping, respectively. A p-doped nanowire 430P and an n-doped nanowire 430N are illustrated in FIG. 22

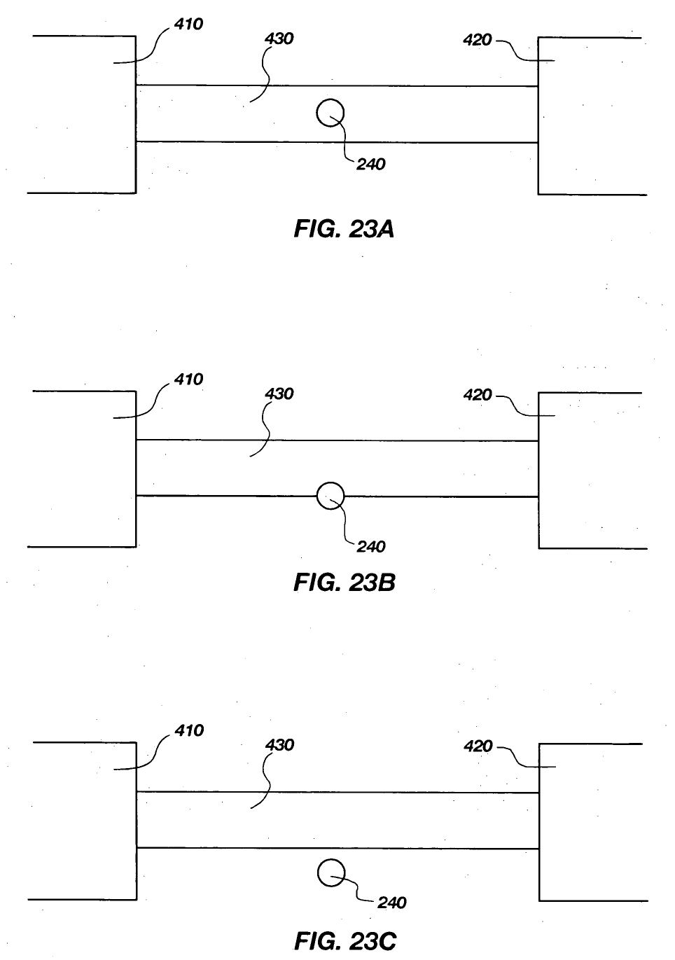

FIGS. 23A through 23C illustrate representative configurations of a nanopore 240 and a nanowire 430. FIG. 23A illustrates a nanopore 240 passing completely through a nanowire 430, FIG. 23B illustrates a nanopore 240 passing partially through a nanowire 430, and FIG. 23C illustrates a nanopore 240 passing substantially near a nanowire 430. As will be apparent to one of skill in the art, any configuration of a nanopore 240 and a nanowire 430, in which nanopore 240 pass wholly through, partially through, or substantially near to nanowire 430, is contemplated within the scope of the invention.

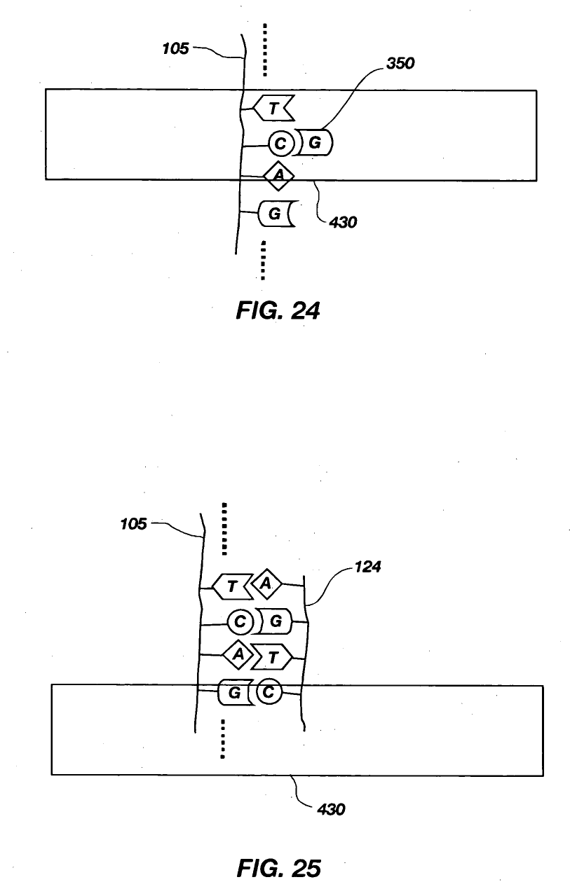

FIG. 24 illustrates a representative molecule sensor, including a nitrogenous material 350 disposed on the nanowire 430. For example, for detecting portions of a polynucleotide chain 105, the nitrogenous material 350 may comprise a base 120 selected from the group consisting of adenine 120A, thymine 120T, uracil 120U, cytosine 120C, and guanine 120G. Furthermore, the nitrogenous material 350 on the nanowire 430 may also include a sugar bonded to the base 120 or a sugar-phosphate bonded to the base 120. By way of example, FIG. 24 illustrates the nitrogenous material 350 guanine 120G. Guanine 120G is illustrated in FIG. 24 as a symbol to show functional interaction with the polynucleotide chain 105. However, it is generally understood that the entire nanowire 430 may be coated with the nitrogenous material 350.

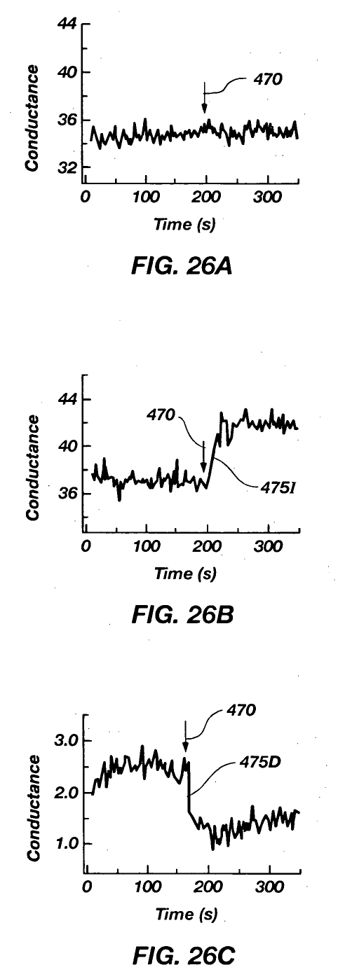

As the polynucleotide chain 105 passes substantially near the coated nanowire 430, a base (in this example, C) of the polynucleotide chain 105 that is complementary to the nitrogenous material 350 (in this example, G) on the nanowire 430 may react with the nitrogenous material 350. This reaction may take the form of a transitory chemical bond between the complementary base on the polynucleotide chain 105 and the nitrogenous material 350 on the nanowire 430. The transitory chemical bond may cause an electronic effect, such as a conductance change 375 (shown in FIGS. 26B and 26C) in the nanowire 430.

FIG. 25 illustrates another representative molecule sensor, including an oligonucleotide 124 attached to the nanowire 430. The oligonucleotide 124 may include many combinations of nucleotides and may be of various lengths to comprise a specific combination of nucleotides that may be of interest. By way of example, FIG. 25 illustrates an oligonucleotide 124 including four nucleotides in the series of C, T, G, and A.

The attachment of the oligonucleotide 124 to the nanowire 430 may be accomplished with a variety of methods known to those of ordinary skill in the art, such as, by way of example only, the methods used in micro-arrays.

As the polynucleotide chain 105 passes near the attached oligonucleotide 124, if a complementary sequence of bases passes near the attached oligonucleotide 124, a transitory chemical bond (i.e., hybridization) may occur between the oligonucleotide 124 and the complementary sequence on the polynucleotide chain 105. In the representative embodiment of FIG. 25, the oligonucleotide 124 comprising the sequence C, T, G, A, may hybridize with the complementary sequence G, A, C, T on the polynucleotide chain 105. As with the single base example of FIG. 24, this transitory chemical bond between the polynucleotide chain 105 and the attached oligonucleotide 124 will cause a conductance change 375 (shown in FIGS. 26B and 26C) of the nanowire 430. A plurality of molecule sensors 300 configured with a variety of oligonucleotides 124 may be useful in determining different specific characteristics of any given polynucleotide chain 105.

The transitory chemical bond results from weak hydrogen bonds between the base 120 (or oligonucleotide 124) on the nanowire 430, and the polynucleotide chain 105. The transitory chemical bond may be broken, allowing continued transportation of the polynucleotide chain 105 by the motive force (e.g. thermal energy, optical energy, or combinations thereof) causing transportation of the polynucleotide chain 105.

FIGS. 26A, 26B, and 26C illustrate measurement of conductance characteristics of the nanowires 430, 430P and 430N previously described with reference to FIG. 22. FIG. 26A illustrates conductance of a p-doped nanowire 430P and an oligonucleotide 124 attached to the p-doped nanowire 430P. An introduction point 470 indicates the point in time where a polynucleotide chain 105 with a non-complementary sequence approaches substantially near the oligonucleotide 124. As can be seen in FIG. 26A, there is no substantial difference in the conductance of the p-doped nanowire 430P.

FIG. 26B illustrates conductance of a p-doped nanowire 430P and an oligonucleotide 124 attached to the p-doped nanowire 430P. An introduction point 370 indicates the point in time where a polynucleotide chain 105 with a complementary sequence approaches substantially near the oligonucleotide 124. When the polynucleotide chain 105 bonds with the base 120 (or oligonucleotide 124) on the p-doped nanowire 430P, the increase of negative charge introduced by the polynucleotide chain 105 enhances the carrier concentration in the p-doped nanowire 430P, resulting in a measurable increase 4751 in the conductance of the p-doped nanowire 430P.

FIG. 26C illustrates conductance of an n-doped nanowire 430N and an oligonucleotide 124 attached to the n-doped nanowire 430N. An introduction point 470 indicates the point in time where a polynucleotide chain 105 with a complementary sequence approaches substantially near the oligonucleotide 124. When the polynucleotide chain 105 bonds with the base 120 (or oligonucleotide 124) on the n-doped nanowire 430N, the increase of negative charge introduced by the polynucleotide chain 105 reduces the carrier concentration in the n-doped nanowire 430N, resulting in a measurable decrease 475D in the conductance of the n-doped nanowire 430N.

Additional electronics may be provided on the substrate, as additional semiconductor devices may be used to sense the conductance change. Also, signal processing hardware (on the substrate or external to the substrate), signal processing software, or a combination thereof, may then be used to gather and process data related to the times when complimentary bases 120 (or complimentary oligonucleotides 124) are substantially near the nanowire 430 and the speed of the polynucleotide chain 105.

Although the foregoing description contains many specifics, these are not to be construed as limiting the scope of the present invention, but merely as providing certain representative embodiments. Similarly, other embodiments of the invention may be devised which do not depart from the spirit or scope of the present invention. Therefore, the scope of the invention is indicated and limited only by the appended claims and their legal equivalents, rather than by the foregoing description. All additions, deletions, and modifications to the invention, as disclosed herein, which fall within the meaning and scope of the claims, are encompassed by the present invention.

What is claimed is:

1. A molecular analysis device, comprising:
at least one molecule sensor, wherein the at least one sensor is selected from the group consisting of a single electron transistor and a nanowire;
at least one nanopore passing at least partially through or substantially near the at least one molecule sensor; and
wherein the at least one molecule sensor develops an electronic effect responsive to a molecule passing through the at least one nanopore.

2. The device of claim 1, wherein the at least one molecule sensor comprises a single electron transistor;
the single electron transistor comprising:
a first terminal;
a second terminal; and
at least one quantum dot positioned between the first terminal and the second terminal; and
wherein the at least one nanopore passes at least partially through or substantially near the at least one quantum dot.

3. The device of claim 2, wherein the electronic effect is a change in electrical charge of the at least one quantum dot indicated by an electrical current change between the first terminal and the second terminal.

4. The device of claim 1, wherein the at least one molecule sensor comprises a single electron transistor;
the single electron transistor comprising:
a first terminal;
a second terminal; and
a nanogap between the first and second terminals; and
wherein the at least one nanopore passes at least partially through or substantially near the nanogap.

5. The device of claim 4, wherein the electronic effect is indicated by an electrical current change between the first terminal and the second terminal.

6. The device of claim 1, wherein the at least one molecule sensor comprises a nanowire that operably couples a first terminal and a second terminal; and wherein the at least one nanopore passes at least partially through or substantially near the nanowire.

7. The device of claim 6, wherein the nanowire is n-type or p-type doped and wherein the electronic effect comprises a measurable change in conductance.

8. The device of claim 1, wherein a nitrogenous material is disposed on at least part of the at least one molecule sensor and is configured for a chemical interaction with an
identifiable configuration of a molecule; and
wherein at least one the molecule sensor develops an electronic effect responsive to the chemical reaction.

9. The device of claim 8, wherein the nitrogenous material comprises material from the group consisting of nucleotides, nucleosides, oligonucleotides, DNA, RNA, amino acids, polypeptides, and proteins.

10. The device of claim 1, wherein the at least one nanopore comprises an entrance point and an exit point; and wherein at least one of the at least one nanopores is configured for substantially straightening the molecule and guiding the molecule at least partially through or substantially near at least one molecule sensors; and
further comprising a transport medium disposed in the at least one nanopore and configured for transporting the molecule in a lengthwise fashion through the at least one nanopore in a transport direction from the entrance point to the exit point to successively present each segment of a plurality of segments distributed along the length of the molecule to the at least one molecule sensor.

11. A method of detecting a molecule, comprising:
guiding at least a portion of a molecule through a nanopore that passes at least partially through or substantially near a molecule sensor, the molecule sensor being selected from the group consisting of a single electron transistor and a nanowire; and
sensing an electronic effect responsive to a molecule passing at least partially through or substantially near the molecule sensor.

12. The method of claim 11, wherein guiding at least a portion of the molecule further comprises transporting the molecule in a transport medium in a lengthwise fashion through the nanopore to successively present each segment of a plurality of segments distributed along the length of the molecule to the molecule sensor.

13. The method of claim 11, wherein a nitrogenous material is disposed on at least part of the molecule sensor and configured for a chemical interaction with an identifiable configuration of a molecule.

14. The method of claim 13, further comprising interacting an identifiable configuration of the molecule and a nitrogenous material disposed on at least part of the molecule sensor; and
sensing an electronic effect responsive to the interaction.

15. The method of claim 13, wherein the nitrogenous material comprises material from the group consisting of nucleotides, nucleosides, oligonucleotides, DNA, RNA, amino acids, polypeptides, and proteins.

16. The method of claim 11, further comprising:
passing the molecule substantially near to at least one additional molecule sensor; and
sensing at least one additional electronic effect responsive to the molecule passing substantially near to the at least one additional molecule sensor.

17. A method of detecting a molecule, comprising:
guiding at least a portion of a molecule through a nanopore that passes at least partially through or substantially near a molecule sensor, the molecule sensor being selected from the group consisting of a single electron transistor and a nanowire; and
sensing an electronic effect responsive to a molecule passing at least partially through or substantially near the molecule sensor;
guiding at least one additional portion of the molecule through at least one additional nanopore that passes at least partially through or substantially near at least one additional molecule sensor, wherein at least one of the additional molecule sensors is selected from the group consisting of a single electron transistor and a nanowire; and
sensing at least one additional electronic effect in the at least one additional molecule sensor responsive to a molecule passing at least partially through or substantially near the at least one additional molecule sensor.

18. The method of claim 17, wherein a nitrogenous material is disposed on at least part of at least one of the at least one additional molecule sensors and configured for a chemical reaction with an identifiable configuration of a molecule; and
wherein the molecule sensor develops an electronic effect responsive to the chemical reaction.

19. The device of claim 18, wherein the nitrogenous material comprises material from the group consisting of nucleotides, nucleosides, oligonucleotides, DNA, RNA, amino acids, polypeptides, and proteins.

20. The method of claim 17, wherein guiding at least a portion of the molecule further comprises transporting the molecule in a transport medium in a lengthwise fashion through the at least one additional nanopore to successively present each segment of a plurality of segments distributed along the length of the molecule to at least one of the at least one additional molecule sensors.
